(19) United States
(12) Patent Application Publication
(10) Pub. No.: US 2022/0118162 A1
(43) Pub. Date: Apr. 21, 2022

(54) BIODEGRADABLE MEMBRANE

(71) Applicant: FRAUNHOFER-GESELLSCHAFT ZUR FÖRDERUNG DER ANGEWANDTEN FORSCHUNG E. V., München (DE)

(72) Inventors: Bastian CHRIST, Würzburg (DE); Sabine AMBERG-SCHWAB, Erlabrunn (DE); Jörn PROBST, Kürnach (DE)

(73) Assignee: FRAUNHOFER-GESELLSCHAFT ZUR FÖRDERUNG DER ANGEWANDTEN FORSCHUNG E. V., München (DE)

(21) Appl. No.: 17/427,999

(22) PCT Filed: Feb. 5, 2020
(86) PCT No.: PCT/EP2020/052789

§ 371 Date: Aug. 3, 2021

(30) Foreign Application Priority Data
Feb. 7, 2019 (DE) 10 2019 201 550.6

(51) Int. Cl. A61L 31/14; A61L 31/12; A61L 31/06; A61L 31/02; A61L 31/16; B29C 41/00

(52) U.S. Cl. CPC A61L 31/148; A61L 31/128; A61L 31/06; A61L 31/028; B29K 2105/0035; B29C 41/003; A61L 2400/12; B29K 2995/006; A61L 31/16

(57) ABSTRACT

The invention relates to a biodegradable membrane on the basis of an organic-inorganic hybrid polymer, and to a process for producing same.

The present invention relates to a biodegradable membrane on the basis of an organic-inorganic hybrid polymer and to a method of producing same.

Adhesions are scarred connective tissue strands that establish an unnatural connection between different body tissues. They can be either congenital or acquired. Acquired adhesions are usually a consequence of surgery. However, they can also arise with inflammatory abdominal diseases or in the context of endometriosis (disease of the lining of the uterus).

Postoperative adhesions are produced despite the best possible surgical technique and tissue-saving surgical methods (such as minimally invasive procedures). They only cause complaints in exceptional cases, but can then substantially impair the health and quality of life of those affected. Adhesions that occur as concomitant effects of surgery in the abdomen and of surgical interventions in the uterus (e.g. curettage or removal of myomas) can be the cause of unwanted childlessness, chronical lower abdominal pain, or a constriction and obstruction of the bowel.

The formation of adhesions is here only a natural reaction of wound healing, e.g. in response to an injury to the peritoneum and/or the organs. According to studies, they are found subsequent to 67 to 93% of all operations in which the peritoneum was opened (G. Pados et al., Prevention of intra-peritoneal adhesions in gynaecological surgery: theory and evidence, in Reproductive BioMedicine Online (2010) 21, 290-303). It can nevertheless occur that organs of the abdomen and of the pelvic area, e.g. intestinal loops, are adhered to one another or to the peritoneum or the fallopian tubes and the ovaries are fettered and fixed in an unnatural manner.

After extensive surgical interventions in the abdomen, e.g. at the bowel, scar tissue is formed that can negatively impair intestinal motility. This occurs in 52-75% of the cases after a colonic resection (approximately 40,000 operations a year in Germany) (see S. Iyer et al., Economic Burden of Postoperative Ileus Associated With Colectomy in the United States, in J Manag Care Pharm. 2009; 15(6):485-94).

There is thus a very great interest in developing a method with which the formation of adhesions can be suppressed or completely prevented.

Known methods are based on the use of different biocompatible barrier materials.

U.S. Pat. No. 7,172,765 B2, for example, describes a process for reducing operative adhesions with the aid of a biodegradable membrane that comprises an electrospun woven fabric without any filler material or matrix material. The manufacture of large areas or amounts of the membrane thus requires a lot of time. It is additionally disadvantageous that cytotoxic solvents are mostly used in electrospinning that subsequently have to be carefully removed.

U.S. Pat. No. 5,948,020 A likewise presents a bioresorbable membrane that can be used in human or animal tissue and prevents the unwanted adhesion of other cells to the tissue connected to the membrane for some time there. The membrane structurally comprises fibers and a polymer matrix. However, only degradable organic polymers are used for the manufacture of both the fibers and the matrix. It is disadvantageous with these that they typically display the effect of shrinkage on contact with physiological media and do not remain stable in shape during the degradation.

Starting from this prior art, it was the object of the present invention to provide a method with which the disadvantages known from the prior art can be overcome and with which membranes can be produced in a simple manner that are biodegradable, but maintain their barrier functions at least over a time period of three to seven days. It was furthermore the underlying object of the present invention to provide a corresponding biodegradable membrane that simultaneously has a high shape stability and a high flexibility.

This object is achieved by the features of claim 1 with respect to the method and by the features of claim 16 with respect to the membrane. Further advantageous embodiments result from the dependent claims.

The method in accordance with the invention of producing a biodegradable membrane on the basis of an organic-inorganic hybrid polymer comprises the following method steps: producing an inorganic sol by stirring a solution containing at least an aqueous solvent, an alkoxy silane, and an acid at a temperature of at least 20° C. (step a); converting the sol into a polymer-sol mixture by adding an end group functionalized biodegradable organic polymer or by adding precursors of the polymer to the sol (step b); and applying the polymer-sol mixture to a front side of a film to harden the mixture to form a hybrid polymer layer (step c), wherein a plurality of fibers are introduced into the mixture in an additional step d) before the hardening of the mixture.

A biodegradable membrane can be produced using this method. Biodegradable is here to be understood such that the membrane degrades on permanent contact with a physiological solution. A buffered aqueous solution that has a pH of 7.3-7.5 can, for example, be considered as the physiological solution. An aqueous PBS solution can in particular be used to check the biodegradability that contains 8.0 g/L NaCl, 0.2 g/L KCl, 1.42 g/L Na₂HPO₄, and 0.27 g/L KH₂PO₄.

The method furthermore permits producing a membrane that is based on two main components. The first component is the organic-inorganic hybrid polymer that serves as the matrix material. The second component is the plurality of fibers that are embedded into the matrix. The properties of both components surprisingly complement one another such that the membrane resulting therefrom has a high tensile strength and simultaneously a high resilience and flexibility.

The organic-inorganic hybrid polymer is here acquired either by addition of an end group functionalized biodegradable organic polymer or by addition of precursors of the polymer to the sol. While the precursors of the polymer are low molecular oligomers that are only precondensed up to a certain degree, the polymer itself is a high molecular polymer.

It has additionally been found that the membrane produced in this manner does not shrink in the first few weeks despite a permanent wetting with a physiological solution and the thereby progressing biodegradation. It initially maintains its original dimensions. It is thereby prevented—with regard to the use of the membrane as an adhesion barrier for avoiding post-operative adhesions of various tissues—that mechanical strains occur during the healing process that could be experienced as unpleasant by patients.

The membrane produced by the method in accordance with the invention furthermore acts dehesively after application onto a cellular tissue. This means that the membrane prevents adjacent cellular tissue from being able to adhere.

Since the production of the polymer-sol mixture can take place as a one-pot synthesis, the method is furthermore suitable for transfer to an industrial scale and for production of larger quantities.

The plurality of fibers in step d) are preferably introduced into the mixture by distribution of fibers on the front side of the film before the application of the mixture (variant i) or by distribution of the fibers on a surface of the applied mixture (variant ii). The fibers here are particularly preferably arranged aligned along a preferred direction or in the form of a regular non-crimp fabric or woven fabric.

These method variants (i and ii) are suitable to produce a membrane in which the fibers are integrated into the hybrid polymer layer produced by hardening the mixture. The decision whether the fibers are aligned along a preferred direction or are arranged in the form of a non-crimp fabric or of a woven fabric should take place while considering the later use. If it is to be expected that the mechanical strain of the membrane is increased in one direction, there is a great indication for aligning the fibers along a preferred direction. An orientation in a preferred direction can be achieved, for example, by spinning the fibers. This can be achieved by the spinning process in that the fibers are placed down in an aligned manner. This can e.g. take place via a programmable charging table that moves at a defined speed in the x and y directions.

It is also possible in the method in accordance with the invention to carry out steps c) and d) multiple times respectively alternately after one another. This has effects on the thickness of the membrane that can be increased by a multiple application of the polymer-sol mixture. In addition, the distribution of the fibers in the cross-section of the membrane can be influenced.

If the fibers are distributed over the film before the application of the mixture on a first-time performance of method steps c) and d) and only after the application of the polymer-sol mixture on the second performance of method steps c) and d), the fiber density on both sides of the membrane is increased on the surface while it is lowered in the interior of the membrane. This is due to the fact that the fibers cannot be distributed uniformly due to the viscosity of the polymer-sol mixture. Different fiber density distributions are reached when a different procedure is followed.

The following procedure is conceivable, for example: A layer of the polymer sol mixture is applied to the film. The fibers are introduced prior to the hardening of the mixture. A layer of the polymer-sol mixture is again applied as the last method step. Fibers are no longer integrated into this layer.

The solution in step a) can be stirred at a temperature of 20 to 50° C., preferably of 30 to 45° C., in particular 40° C. A stirring time of at least 7 hours is preferred, particularly preferably of at least 8 hours, in particular of at least 18 hours.

These method parameters ensure that the alkoxy silane is converted into polysiloxane and a sol is formed in this manner.

It is further preferred if the mixture in step c) is applied by spread coating or by flooding the front side of the film, with the spread coating preferably taking place with the aid of a doctor blade, a spiral, or of a film drawing device.

The alkoxy silane can be selected from the group consisting of tetramethyl orthosilicate, tetraethyl orthosilicate, tetrapropyl orthosilicate, tetraisopropyl orthosilicate, tetrabutyl orthosilicate, and mixtures thereof.

The end group functionalized biodegradable organic polymer is preferably selected from the group consisting of end group functionalized polyesters, end group functionalized polyalcohols, end group functionalized polyoxazolines, end group functionalized polyanhydrides, end group functionalized polysaccharides, end group functionalized polyhydroxyalkanoates, end group functionalized proteins, and mixtures thereof. The end group functionalized biodegradable organic polymer is preferably an elastomer. The end group functionalization preferably comprises terminal hydroxyl groups, carboxylic acid groups, thiol groups, amine groups, epoxy groups, and/or trialkoxy silane groups, particularly preferably terminal hydroxyl groups. A well-suited end group functionalized biodegradable organic polymer is silanized polycaprolactam (PCL).

The end group functionalization of the organic polymer ensures that it is at least partially covalently connected to the inorganic sol.

The structure of the organic polymers is as desired here. Both hybrid polymers having linear organic polymers and having branched or star-shaped organic polymers can be formed. The solubility of the organic polymers is more important. It is preferred here that the organic polymers dissolve well in an aqueous or alcoholic solution or in a mixture of water and alcohol, or can at least be finely dispersed therein. Biodegradable elastomers are particularly preferably used as the organic polymers.

It is advantageous for the acid used in method step a) to be a sulfonic acid, preferably methanesulfonic acid. The acid can, hover, also comprise a mineral acid, e.g. HCl, H₂SO₄, HNO₃, HI or H₃PO₄. Mixtures of the aforesaid acids are equally possible. The production of the biodegradable membrane is additionally facilitated when the solution in step a) of the method has a pH between 1 and 7, preferably between 1 and 3.

Water or a mixture of water and an alcohol, tetrahydrofurane or toluoyl can be considered as the aqueous solvent. Of these, the mixture of water and an alcohol, e.g. ethanol, is to be particularly recommended. Such a mixture makes it possible to produce the membrane without the use of solvents that are dangerous to the health. A careful separation of solvent residues is thus not necessary.

The polymer in step b) of the method is preferably added in a weight ratio of 0.1 to 10.0, particularly preferably of 0.5 to 8.0, in particular of 1.0 to 6.0, with respect to the mass of the sol added in step a). It is ensured in this manner that the parts by weight of the organic polymer in the hybrid polymer layer amount to at least 10 wt %, preferably at least 20 wt %. It is particularly preferred if the parts by weight of the organic polymer in the hybrid polymer layer are between 33 and 85 wt %.

Biodegradable fibers of any kind can be used as the fibers. They can be of an organic, inorganic, or hybrid nature. The length of the fibers can also vary; endless fibers, long fibers, but also shorter fiber pieces can also be integrated in the polymer-sol mixture.

Fibers are preferably used from the group comprising silica gel fibers, fibers containing TiO₂, polyester fibers, polyanhydride fibers, polysaccharide fibers, polyhydroxyalkanoate fibers, protein fibers, and mixtures thereof, preferably from the group comprising silica gel fibers and fibers containing TiO₂, with the fibers particularly preferably being used in a proportion of a maximum of 50 wt %, very particularly preferably of a maximum of 33 wt %, in particular a maximum of 25 wt %, with respect to the total mass of the hybrid polymer layer and fibers. Silica gel fibers have the advantage that they are structurally very similar to the inorganic sol that is produced from the alkoxy silane and can thereby easily be integrated into the polymer-sol mixture.

Particularly tear-proof membranes are obtained when fibers are used in the method in accordance with the invention having a tensile strength of at least 2800 Mpa, preferably of at least 2950 Mpa, in particular of at least 3000 Mpa.

The fibers preferably have a diameter of 1 nm to 2 mm, preferably of 1 to 100 μm, in particular of 50-60 μm.

In accordance with a further preferred embodiment, the fibers are fibers obtained through the method of electrospinning, preferably fibers obtained through the method of electrospinning selected from the group consisting of silica gel fibers, fibers containing TiO₂, polyester fibers, polyanhydride fibers, polysaccharide fibers, polyhydroxyalkanoate fibers, protein fibers, and mixtures thereof. If silica gel is used as the material for these fibers, the fibers preferably have a diameter in the range from 100 nm to 5 μm, particularly preferably in the range from 500 nm to 2 μm.

In a variant of the method, the membrane is released from the front side of the film after a drying time of at least 30 minutes, with the front side of the film preferably being dehesive and/or comprising ethylene tetrafluoroethylene copolymer.

An embodiment of the method that is particularly useful from a medical aspect is obtained when at least one pharmacologically active compound is added in step a) or b) of the method or is used to impregnate the hybrid polymer layer after the hardening, with the proportion of the pharmacologically active compound being selected such that it preferably amounts to 1 to 20 wt % with respect to the total mass of the hybrid polymer layer and the fibers.

In particular antibiotics and substances that reduce scarring can be considered as the pharmacologically active compound. These compounds can be released during the degradation of the membrane. They can be integrated into the membrane either as encapsulated or in pure form. An encapsulation can take place by formation of micelles, liposomes with the aid of block copolymers, or inorganic particulate systems such as mesoporous or microporous particles. If pharmacologically active compounds are integrated into the membrane, the temperature during production may by no means exceed the degradation temperature of the active ingredient.

A membrane is furthermore provided from a biodegradable organic-inorganic hybrid polymer and a plurality of fibers.

The membrane can be disintegrated and/or degraded by contact with a physiological solution, preferably with an aqueous PBS solution comprising NaCl, KCl, Na₂HPO₄ and KH₂PO₄. The membrane is particularly preferably degraded to at least 35 wt % over its total mass, preferably to at least 60 wt % of its total mass, within 64 days on wetting with such a physiological solution.

After 8 days of contact with an aqueous PBS solution, the membrane in accordance with the invention is preferably disintegrated and/or degraded to at least 10 wt % of its total mass, preferably to at least 20 wt % of its total mass, particularly preferably to at least 25 wt % of its total mass.

The membrane in accordance with the invention is furthermore characterized in that it forms a barrier for a growth or an adhesion of human or animal cells over a time period of at least 3 days, preferably 5 days, particularly preferably 7 days.

The membrane is produced in accordance with the initially described method in accordance with the invention.

It is also conceivable here that the membrane is provided or printed with additional layers in this method.

The possibilities for use of the membrane in accordance with the invention are manifold.

Apart from in vivo applications, the membrane can be used for substance separation, in particular as a filtration membrane. Filtration membranes having active functions whose separation performance can be set in a targeted manner by the polarity of the matrix and the design of the hybrid film structure can be realized with the aid of the membrane.

On the other hand, the membrane in accordance with the invention can be used in surgical interventions in which there is the risk of postoperative formation of adhesions or scarring, in particular on the insertion of prostheses and implants or as active ingredient carriers in pharmaceutical processes.

In surgical interventions, the membrane serves as an adhesion barrier or as a mechanical barrier and prevents cells from adjacent tissues accumulating at that tissue to which the membrane is attached. An effective reduction of scarring is thereby possible, which is particularly relevant in esthetic surgery.

However, the use of the membrane in the insertion of prostheses and implants also has great potential since it results in an undisturbed and thus optimized ingrowth of the implants.

A pharmaceutical/therapeutic active ingredient patch based on the membrane can in contrast be considered as an environmentally friendly active ingredient carrier that can be simply composted after use.

The use of the membrane is also conceivable in biodegradable sensors, biodegradable active implants, to cover open wounds, and in the field of tissue engineering and cell cultivation.

The invention will be explained in more detail with reference to the following examples and Figures. They are here only to be understood by way of example and should not restrict the scope of the invention.

Trial protocols are given in Examples 1 and 2 according to which a membrane in accordance with the invention can be produced. In Methods 1 to 3, variants of Example 1 are described that show the manner in which the membrane in accordance with the invention can still be realized starting from the polymer-sol mixture and the fibers.

### EXAMPLE 1

5 mol tetraethoxy silane (TEOS) and 9.6 mol ethanol are presented in a 2 liter round bottom flask and are mixed at 40° C. at 200 r.p.m. in a polyethylglycol bath. 160 g of a 0.1 N-methanesulfonic acid solution are subsequently dripped in. The reaction mixture was thereupon heated to 40° C. for one day at a stirring speed of 200 r.p.m. 22 mol ethanol was withdrawn with the aid of a rotary evaporator. The mass of the flask content was determined and twice the mass of silanized polycaprolactone triol (manufactured according to Liu et al., Journal of Applied Polymer Science 109 (2008), p. 1105-1113) was stirred in. This polymer-sol mixture will be called “Mixture 1” in the following.

After stirring at room temperature for 30 min, silica gel fibers (manufactured according to Emmert et al., RSC Adv., 2017, 7, 5708) having a diameter of 50 μm were presented on an ETFE film and were flooded with Mixture 1. The ETFE substrate was here slanted at an angle of 25° so that the fluid is distributed over the fibers. The dried film was pulled off the ETFE film as a membrane after one day.

### EXAMPLE 2

5 mol tetraethoxy silane (TEOS) and 9.6 mol ethanol are presented in a 2 liter round bottom flask and are mixed at 40° C. at 200 r.p.m. in a polyethylglycol bath. 160 g of a 0.1 N-methanesulfonic acid solution are subsequently dripped in. The reaction mixture was thereupon heated to 40° C. for one day at a stirring speed of 200 r.p.m. 22 mol ethanol was withdrawn with the aid of a rotary evaporator. The mass of the flask content was determined and five times the mass of silanized polycaprolactone triol (manufactured according to Liu et al., Journal of Applied Polymer Science 109 (2008), p. 1105-1113) was stirred in. This polymer-sol mixture will be called “Mixture 2” in the following.

After stirring at room temperature for 30 min, fibers (manufactured according to Emmert et al., RSC Adv., 2017, 7, 5708 “Nanostructured surfaces of biodegradable silica fibers enhance directed amoeboid cell migration in a microtubule-dependent process”) having a diameter of 50 μm are presented on an ETFE film and are spread coated with the produced Mixture 2 with the aid of a film drawing device. The lacquer film spreader had a thickness of 225 μm. The dried film was pulled off the ETFE film as a membrane after one day.

### EXAMPLE 3

A Mixture 2 is prepared analogously to Example 2. The mixture is subsequently coated by a doctor blade over the fibers presented on an ETFE film in accordance with Method 3.

Method 1:

Mixture 1 is applied on a film in a liquid-viscous state by a doctor blade. Fibers are then applied onto the layer coated by a doctor blade and a further layer is then distributed over the fibers by a doctor blade. After a certain wetting time and after evaporation of solvents, the fiber reinforced film can be pulled off the substrate.

Method 2:

Mixture 1 is applied on a film in a liquid-viscous state by a doctor blade. Fibers are then applied to the layer coated by a doctor blade and Mixture 1 is flooded over the fibers. After a certain wetting time and after evaporation of solvents, the fiber reinforced film can be pulled off the substrate.

Method 3:

Fibers are presented on a film and Mixture 1 is applied in a liquid-viscous state by a doctor blade. After a certain wetting time and after evaporation of solvents, the fiber reinforced film can be pulled off the substrate.

Mixtures 1 and 2 can generally be applied using any of the Methods 1 to 3.

There are furthermore shown:

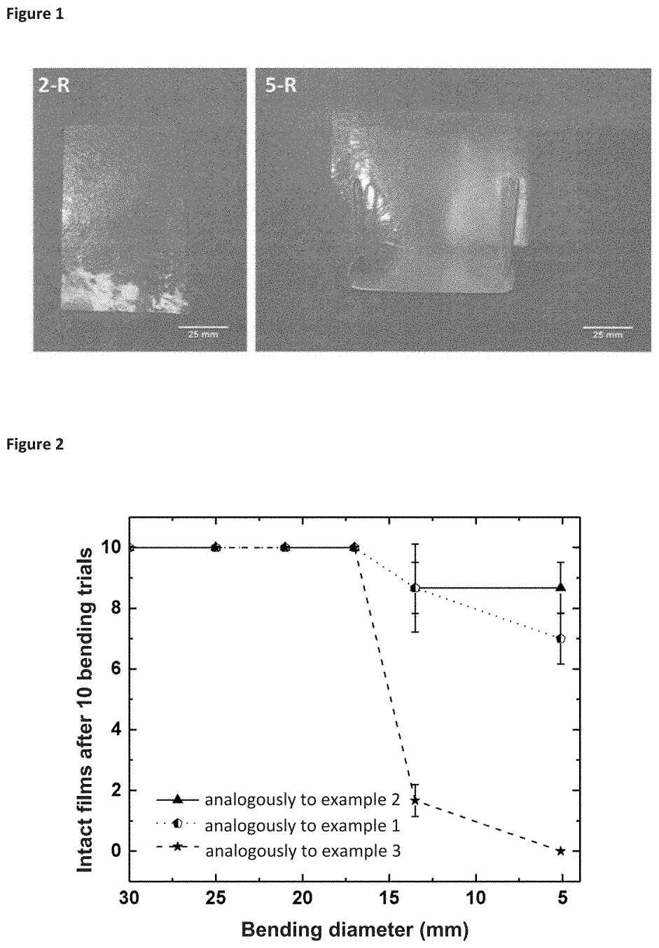

FIG. 1: a photograph of two membranes in accordance with the invention;

FIG. 2: a diagram on the resilience of membranes in accordance with the invention;

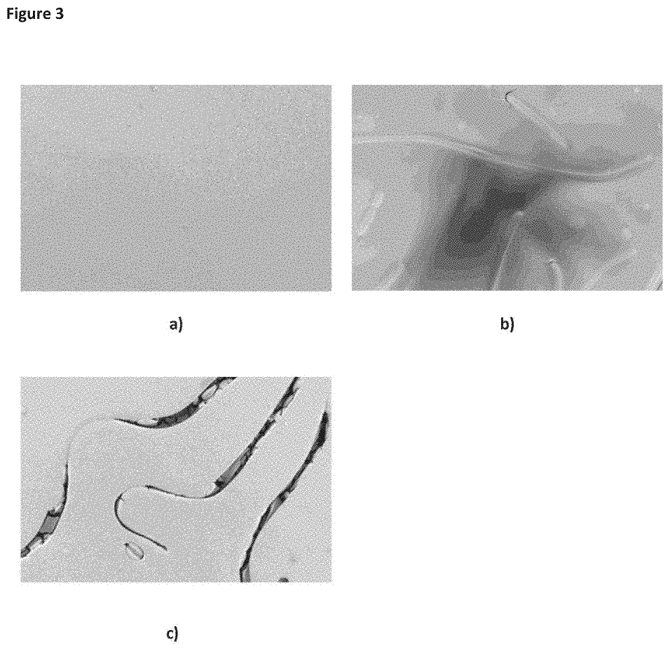

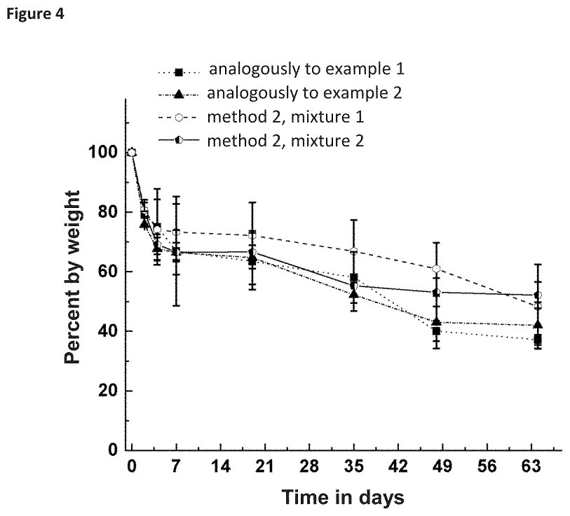

FIG. 3: SEM photographs on the degradation of membranes in accordance with the invention;

FIG. 4: degradation profiles of different membranes; and

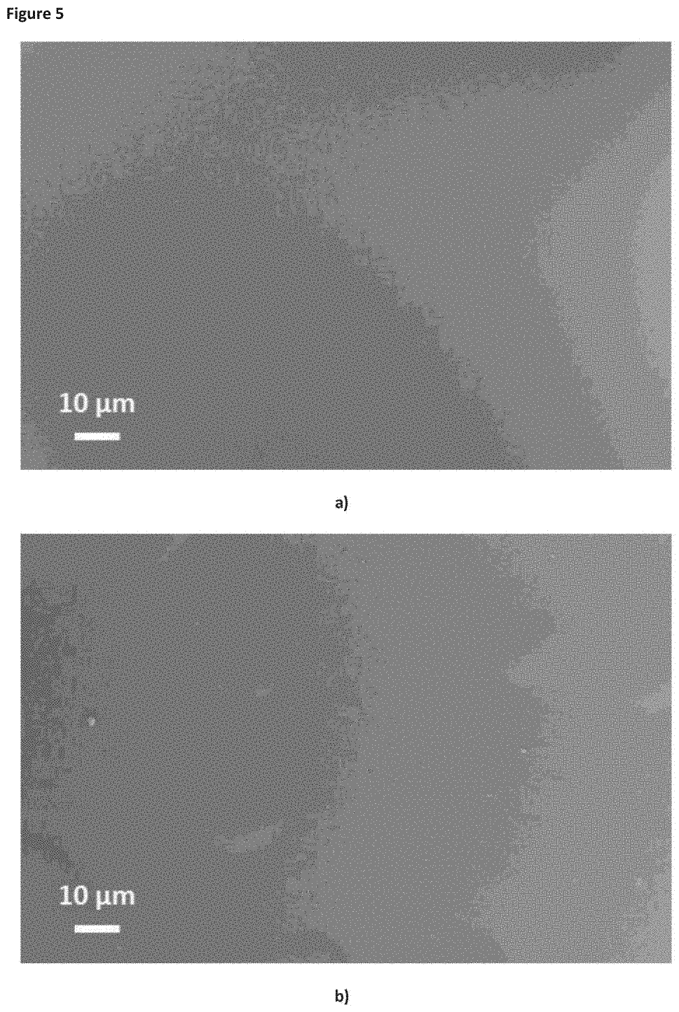

FIG. 5: SEM photographs of surfaces of membranes in accordance with the invention.

FIG. 1 shows fiber reinforced membranes that were cut to size from a DIN A4 film and that were already removed from the substrate. They are membranes that were produced in accordance with Example 1 (2-R) and Example 2 (5-R). The fiber reinforced membrane is intrinsically stable, flexible, and has a homogeneous structure.

FIG. 2 shows a diagram from which it can be seen that the resilience of the membranes produced in accordance with the invention is improved with the increasing proportion of organic polymer in the hybrid polymer layer. If twice the mass or five times the mass of organic polymer relative to the inorganic sol is used in the production process, the resulting membrane also remains intact after multiple bending with a small bending diameter. If the mass ratio of organic polymer to inorganic sol in the hybrid polymer layer is only 1:1, approximately 75-90% of all membranes rupture after 10× folding with a bending diameter of 14 mm. The trial results shown in FIG. 2 here relate exclusively to the membranes that, in accordance with the invention, are fiber reinforced and are biodegradable. Membranes that are not fiber reinforced cannot be handled. They rupture so easily that no bending trials at all could be performed.

FIG. 3 shows three SEM photos a), b), and c) of a membrane that has been produced in accordance with Example 1 and has subsequently been wetted with a physiological solution. The film surface is still very homogeneous in FIG. 3a, that shows a non-degraded film. After 7 days, the film surface gradually dissolves such that the integrated fibers are exposed (cf. photograph in FIG. 3b). After 64 days, the fibers have been removed from the membrane (cf. photograph in FIG. 3c). The membrane has here, however, not contracted, but has retained its original shape. The degradation of the membrane does not, however, progress further even after 64 days (not shown).

FIG. 4 shows degradation profiles of different membranes in accordance with the invention in a phosphate-buffered saline solution over a period of 64 days. The rectangular measurement points represent a membrane that was produced in accordance with Example 1. The triangular measurement points were found with a membrane that was produced in accordance with Example 2. The degradation data of the membranes that were produced in accordance with Method 2 using Mixtures 1 and 2 are behind the pentagonal and hexagonal measurement points respectively.

FIG. 5 shows SEM photographs of surfaces of two membranes in accordance with the invention. They are largely transparent for visible light and have a smooth surface structure.

1-21. (canceled)

22. A method of producing a biodegradable membrane from an organic-inorganic hybrid polymer, the method comprising:
(a) producing an inorganic sol by stirring a solution containing at least an aqueous solvent, an alkoxy silane, and an acid at a temperature of at least 20° C.;
(b) converting the sol to a polymer-sol mixture by addition of an end group functionalized biodegradable organic polymer or by addition of a precursor of the polymer to the sol; and
(c) applying the polymer-sol mixture to a front side of a film and hardening the mixture to form a hybrid polymer layer,
wherein, in an additional step (d), a plurality of fibers are introduced into the mixture before hardening the mixture.

23. The method in accordance with claim 22, wherein the plurality of fibers are introduced into the mixture in step (d) by:
(i) distributing fibers on the front side of the film before the application of the mixture; or
(ii) distributing the fibers on a surface of the applied mixture.

24. The method in accordance with claim 22, wherein steps (c) and (d) are performed multiple times respectively alternately after one another.

25. The method in accordance with claim 22, wherein the solution in step (a) is stirred at a temperature of 20 to 50° C.

26. The method in accordance with claim 22, wherein the polymer-sol mixture is stirred between step (b) and step (c) for a duration of 0.5 minutes to 45 minutes.

27. The method in accordance with claim 22, wherein the mixture in step (c) is applied by spread coating or by flooding the front side of the film.

28. The method in accordance with claim 22, wherein the alkoxy silane is selected from the group consisting of tetramethyl orthosilicate, tetraethyl orthosilicate, tetrapropyl orthosilicate, tetraisopropyl orthosilicate, tetrabutyl orthosilicate, and mixtures thereof.

29. The method in accordance with claim 22, wherein the end group functionalized biodegradable organic polymer is selected from the group consisting of end group functionalized polyesters, end group functionalized polyalcohols, end group functionalized polyoxazolines, end group functionalized polyanhydrides, end group functionalized polysaccharides, end group functionalized polyhydroxyalkanoates, end group functionalized proteins, and mixtures thereof.

30. The method in accordance with claim 22, wherein the acid is a sulfonic acid, a mineral acid, or a mixture thereof, and/or the solution in step (a) has a pH between 1 and 7.

31. The method in accordance with claim 22, wherein the aqueous solvent is water or a mixture of water and an alcohol, tetrahydrofuran, or toluene.

32. The method in accordance with claim 22, wherein the polymer in step (b) is added to the inorganic sol in a weight ratio of 0.1 to 10.0.

33. The method in accordance with claim 22, wherein the fibers are selected from the group consisting of silica gel fibers, fibers containing TiO₂, polyester fibers, polyanhydride fibers, polysaccharide fibers, polyhydroxyalkanoate fibers, protein fibers, and mixtures thereof.

34. The method in accordance with claim 22, wherein the fibers have a diameter of 1 nm to 2 mm.

35. The method in accordance with claim 22, wherein the membrane is released from the front side of the film after a drying time of at least 30 minutes.

36. The method in accordance with claim 22, wherein at least one pharmacologically active compound is added in step (a) or (b) or is impregnated into the hybrid polymer layer after the hardening.

37. A membrane comprising a biodegradable organic-inorganic hybrid polymer and a plurality of fibers.

38. The membrane in accordance with claim 37, wherein the membrane disintegrates and/or degrades when contacted with a physiological solution.

39. The membrane in accordance with claim 37, which, after 8 days of contact with an aqueous PBS solution, the membrane disintegrates or degrades to at least 10 wt % of its total mass.

40. A barrier for growth of human or animal cells comprising a membrane in accordance with claim 37, which forms a barrier for growth for a period of at least 3 days.

41. A membrane produced in accordance with a method of claim 22.

42. A method of surgically preventing formation of adhesion or scarring in a postoperative patient, comprising applying a membrane of claim 37 to the patient postoperatively.
